(19) United States
(12) Patent Application Publication
(10) Pub. No.: US 2022/0229049 A1
(43) Pub. Date: Jul. 21, 2022

(54) REAGENTS FOR SANDWICH IMMUNOASSAYS USING PARTICLE ENHANCED AGGLUTINATION DETECTION AND METHODS OF PRODUCTION AND USE THEREOF

(71) Applicant: Siemens Healthcare Diagnostics Inc., Tarrytown, NY (US)

(72) Inventor: Tie Wei, Wilmington, DE (US)

(73) Assignee: Siemens Healthcare Diagnostics Inc., Tarrytown, NY (US)

(21) Appl. No.: 17/595,478

(22) PCT Filed: Oct. 25, 2019
(86) PCT No.: PCT/US2019/058068

§ 371 Date: Nov. 17, 2021

Related U.S. Application Data
(60) Provisional application No. 62/868,309, filed on Jun. 28, 2019.

(51) Int. Cl. G01N 33/53; G01N 33/543

(52) U.S. Cl. CPC G01N 33/5308; G01N 2470/04; G01N 33/54313

(57) ABSTRACT

Reagents for use in sandwich immunoassays for haptens are disclosed, along with kits containing same. Also disclosed are diagnostic immunoassay methods that utilize these immunoassay reagents in a particle enhanced agglutination detection assay for detecting haptens/drugs in samples.

### CROSS REFERENCE TO RELATED APPLICATIONS/INCORPORATION BY REFERENCE STATEMENT

The subject application claims benefit under 35 USC § 119(e) of U.S. provisional Application No. 62/868,309, filed Jun. 28, 2019. The entire contents of the above-referenced patent application are hereby expressly incorporated herein by reference.

### STATEMENT REGARDING FEDERALLY SPONSORED RESEARCH OR DEVELOPMENT

Not Applicable.

### BACKGROUND

The body relies upon a complex immune response system to distinguish self from non-self. At times, the body's immune system must be controlled in order to either augment a deficient response or suppress an excessive response. For example, when organs such as kidney, heart, heart-lung, bone marrow, and liver are transplanted in humans, the body will often reject the transplanted tissue by a process referred to as allograft rejection.

In treating allograft rejection, the immune system is frequently suppressed in a controlled manner with drug therapy. Immunosuppressant drugs are therapeutic drugs that are carefully administered to transplant recipients in order to help prevent allograft rejection of non-self tissue. Immunosuppressive drugs include (but are not limited to): glucocorticoids, cytostatics, antibodies, drugs acting on immunophilins, and other drugs such as (but not limited to) interferons, opiates INF binding proteins, mycophenolate, FTY720, and the like. A particular class of immunosuppressant drugs comprises those drugs that act on immunophilins. Immunophilins are an example of high-affinity, specific binding proteins having physiological significance. Two distinct families of immunophilins are presently known: cyclophilins and macrophilins, the latter of which specifically bind, for example (but not by way of limitation), tacrolimus, sirolimus, or everolimus.

Two most commonly administered immunosuppressive drugs to prevent organ rejection in transplant patients are cyclosporine (CSA) and tacrolimus (FK506). Another drug that finds use as an immunosuppressant in the United States and other countries is sirolimus, also known as rapamycin. Derivatives of sirolimus are also useful as immunosuppressants; such derivatives include, for example (but not by way of limitation), everolimus and the like.

Tacrolimus, also known as FK506, is a cyclic, poly-N-methylated undecapeptide that possesses immunosuppressive activity and that is isolated from the fermentation product of the bacteria Streptomyces tsukubaensis No 9993. The structure of FK506 is shown in Formula I below.

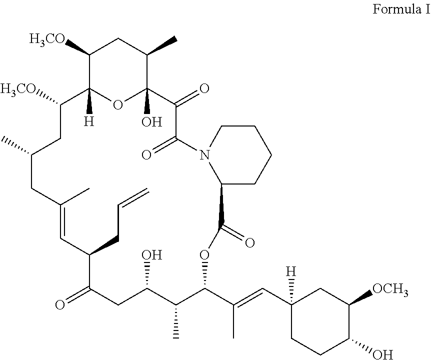 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

Tacrolimus is commonly used, often along with other immunosuppressant drugs, to reduce graft rejection in allogeneic organ transplants by suppressing the immune system. Tacrolimus has a narrow therapeutic window, and thus it is critical to monitor blood drug concentrations for optimal efficacy. For tacrolimus drug monitoring, competitive immunoassays employing a single antibody are commercially available, and a sandwich immunoassay, which should offer higher analytical sensitivity and specificity and wider dynamic ranges than the competitive format, has been described (Wei et al., Clinical Chemistry (2014) 60 (4):621-630; and U.S. Pat. No. 8,586,322).

Sirolimus, also known as rapamycin, is a macrolide antibiotic produced by Streptomyces hygroscopicus, and has been found to be pharmaceutically useful in a variety of applications, particularly as an immunosuppressant, e.g., for use in the treatment and prevention of organ transplant rejection and autoimmune diseases. The structure of sirolimus (rapamycin) is shown in Formula II below.

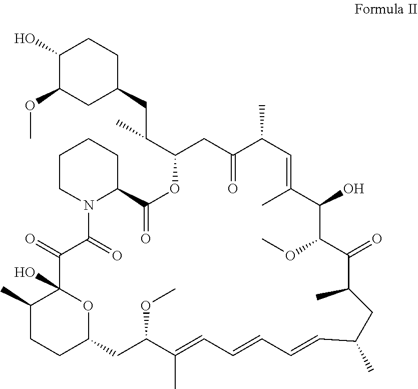 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Sirolimus, however, does exhibit side effects at higher dosages, and it has a somewhat variable bioavailability. Monitoring blood levels of rapamycin in patients being treated with rapamycin is thus very desirable in order to be able to regulate the dosage so as to maintain the minimum level sufficient for pharmacologic activity and to avoid any undue risk of side effects. Rapamycin assays have recently been described in U.S. Pat. Nos. 6,635,745; 8,039,599; and 8,039,600.

Everolimus [40-O-(2-hydroxyethyl)-rapamycin], also known as SDZ-RAD, RAD, and CERTICAN® (Novartis), is a novel macrolide immunosuppressant that was developed by Novartis (Nashan, B., Transplantation Proceedings (2001) 33: 3215-3230) in an effort to improve upon sirolimus. Everolimus has greater stability and enhanced solubility in organic solvents, as well as more favorable pharmokinetics with fewer side effects, than sirolimus. The structure of everolimus is shown in Formula III below.

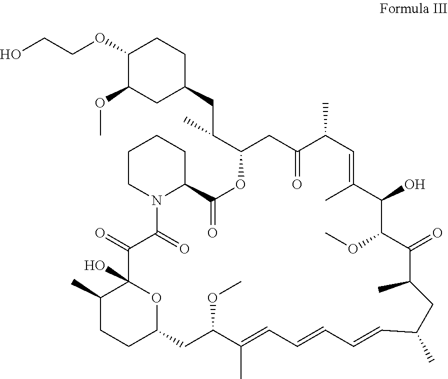 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

However, there exists a similar need for therapeutic drug monitoring (TDM) with everolimus as with tacrolimus and sirolimus. Immunoassays for everolimus are disclosed, for example, in U.S. Pat. No. 7,223,553.

As stated herein above, the side effects associated with these immunosuppressant drugs can be controlled in part by carefully controlling the level of the drug present in a patient. Therapeutic monitoring of concentrations of immunosuppressant drugs and related drugs in biological samples is required to optimize dosing regimens to ensure maximal immunosuppression with minimal toxicity. Although immunosuppressant drugs are highly effective immunosuppressive agents, their use must be carefully managed, because the effective dose range is often narrow, and excessive dosage can result in serious side effects. On the other hand, too little dosage of an immunosuppressant can lead to tissue rejection. Because distribution and metabolism of an immunosuppressant drug can vary greatly between patients, and because of a wide range and severity of adverse reactions, accurate monitoring of the drug level is essential.

Small hapten drug/hormone assays (such as, but not limited to, tacrolimus assays) are often measured by competitive immunoassays that use a single antibody. This type of assay often employs particle enhanced agglutination for signal detection. However, these assays have drawbacks. First, these competitive assays typically have low sensitivity and are usually less precise than sandwich assays. Second, low specificity, cross-reactivity with structural analogs is higher than a sandwich assay which requires recognition of more epitopes. Third, it is harder to remove sample matrix in the reaction mixture in the presence of hapten analog(s); for this reason, removing the sample matrix in such assays often requires the use of manual extraction procedures.

In addition, sandwich assays for small haptens have been reported that utilize two antibodies specific for the hapten (see U.S. Pat. No. 8,586,322). However, the use of particle enhanced agglutination to formulate a sandwich assay for haptens has not been reported previously.

Therefore, there is a need in the art for new and improved immunoassays for analytes (such as, but not limited to, haptens) that overcome the disadvantages and defects of the prior art. It is to such immunoassays, as well as reagents and kits used therein, that the present disclosure is directed.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, kits, and/or methods disclosed herein can be made and executed without undue experimentation in light of the present disclosure. While the compositions, kits, and/or methods have been described in terms of particular embodiments, it will be apparent to those skilled in the art that variations may be applied to the compositions, kits, and/or methods and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The terms “analog” and “derivative” are used herein interchangeably and refer to a substance which comprises the same basic carbon skeleton and carbon functionality in its structure as a given compound, but can also contain one or more substitutions thereto. The term “substitution” as used herein will be understood to refer to the replacement of at least one substituent on a compound with a residue R. In certain non-limiting embodiments, R may include H, hydroxyl, thiol, a halogenid selected from fluoride, chloride bromide or iodite, a C1-C4 compound selected one of the following: linear, branched or cyclic alkyl, optionally substituted, and linear branched or cyclic alkenyl, wherein the optional substitutents are selected from one or more alkenylalkyl, alkynylalkyl, cycloalkyl, cycloalkenylalkyl, arylalkyl, heteroarylalkyl, heterocyclealkyl, optionally substituted heterocycloalkenylalkyl, arylcycloalkyl, and arylheterocycloalkyl, each of which is optionally substituted wherein the optional substitutents are selected from one or more of alkenylalkyl, alkynylalkyl, cycloalkyl, cyclalkenylalkyl, arylalkyl, alkylaryl, heteroarylalkyl, heterocyclealkyl, optionally substituted heterocycloalkenylalkyl, arylcycloalkyl, and arylheterocyclalkyl, phenyl, cyano, hydroxyl, alkyl, aryl, cycloalkyl, cyano, alkoxy, alkylthio, amino, —NH (alkyl), —NH(cycloalkyl)₂, carboxy, and —C(O))-alkyl.

The term “specific binding partner,” as used herein, will be understood to refer to any molecule capable of specifically associating with a macrophilin-binding pharmaceutical for purposes of detection thereof. For example, but not by way of limitation, the specific binding partner may be an antibody, a receptor, a ligand, an aptamer, a molecular imprinted polymer (i.e., inorganic matrices), or any combination and/or derivative(s) thereof, as well as any other molecules capable of specific binding to the macrophilin-binding pharmaceutical.

The term “antibody” is used herein in the broadest sense and refers to, for example, intact monoclonal antibodies and polyclonal antibodies, multi-specific antibodies (e.g., bispecific antibodies), antibody fragments and conjugates thereof that exhibit the desired biological activity of analyte binding (such as, but not limited to, Fab, Fab′, F(ab′)2, Fv, scFv, Fd, diabodies, single-chain antibodies, and other antibody fragments and conjugates thereof that retain at least a portion of the variable region of an intact antibody), antibody substitute proteins or peptides (i.e., engineered binding proteins/peptides), and combinations or derivatives thereof. The antibody can be of any type or class (e.g., IgG, IgE, IgM, IgD, and IgA) or sub-class (e.g., IgG1, IgG2, IgG3, IgG4, IgA1, and IgA2).

The term “sample” as used herein will be understood to include any type of biological sample that may be utilized in accordance with the present disclosure. In certain embodiments, the sample may be any fluidic sample and/or sample capable of being fluidic (e.g., a biological sample mixed with a fluidic substrate). Examples of biological samples that may be utilized include, but are not limited to, whole blood or any portion thereof (i.e., plasma or serum), saliva, sputum, cerebrospinal fluid (CSF), surgical drain fluid, skin, intestinal fluid, intraperitoneal fluid, cystic fluid, sweat, interstitial fluid, extracellular fluid, tears, mucus, bladder wash, urine, swabs, semen, fecal, pleural fluid, nasopharyngeal fluid, combinations thereof, and the like. It should be noted that although the present disclosure is directed towards a biological sample, one skilled in the art will appreciate that the concepts disclosed herein may be applied to any sample wherein a concentration of an analyte (such as, but not limited to, a hapten) may be determined, and as such, the scope of the present disclosure is not limited to biological samples.

Turning now to the presently disclosed and/or claimed inventive concept(s), reagents utilized in sandwich immunoassays for analytes (such as, but not limited to, haptens) using particle enhanced agglutination detection, as well as kits containing same and methods of production and use thereof, are disclosed that overcome the disadvantages and defects of the prior art. The presently disclosed and/or claimed inventive concept(s) combines a unique antibody coating design on particles with the sandwich assay format to maximize the assay signal while reducing the number of reagents required to only two reagents. For example, a typical sandwich immunoassay requires the manufacture of five different reagents: a pretreatment (or extraction) reagent, a labeled antibody (i.e., a FITC antibody), a solid phase (i.e., a magnetic particle coated with anti-FITC antibody), ancillary reagent (i.e., fluorescein labeled second antibody), and wash and signal generation solutions. In contrast, the immunoassays described herein only require the manufacture of two reagents. In one non-limiting embodiment, the two reagents include a pretreatment reagent and a single particle coated with two different antibodies. In another non-limiting embodiment, the two reagents include a pretreatment reagent and a reagent comprising a mixture of two particles, each coated with one of the two antibodies. In yet another non-limiting embodiment, the two reagents includes a pretreatment reagent and a reagent comprising a particle with an antibody attached thereto and a second antibody in its “free” form (i.e., not attached to a particle).

The immunoassay formats of the present disclosure will give higher assay sensitivity as well as lower cross-reactivity with drug analogs than a competitive immunoassay format. In addition, the immunoassays described herein can be fully automated and do not require any manual extraction steps. Only two reagents, a pretreatment reagent and a particle-dual antibody reagent, are needed for the immunoassay format disclosed herein. As such, the presently disclosed and/or claimed inventive concept(s) includes the first sandwich assay format for haptens that utilizes particle enhanced agglutination for signal detection. In addition, the presently disclosed and/or claimed inventive concept(s) includes the first assay to coat latex particles with a mixture of two antibodies to form inter-particle sandwiches for agglutination. Further, the presently disclosed and/or claimed inventive concept(s) includes the first assay where particle agglutination is bridged by a hapten.

One of major advantages of the particle enhanced agglutination assay over other technologies (such as those that utilize enzyme or chemiluminescent tags) is simplification of reagent preparation. For example, only one particle reagent is needed for the assay; if both anti-analyte antibodies (such as, but not limited to, anti-hapten antibodies) are conjugated to the same latex particles, agglutination occurs when the analyte/hapten bridges the particles. Another example is that if one antibody is conjugated to the latex particle, the other antibody can be used directly in the assay to trigger agglutination via the particle-bound analyte/hapten without requiring conjugation of the second antibody to a particle. Another major advantage of the presently disclosed immunoassay design is simplification of the assay process. The particle enhanced signal detection utilized herein allows one immuno-reagent (as in the case where both antibodies are attached the same particles) or a mixture of the antibody coated particle and free antibody as one reagent. Assay formats using enzyme or chemiluminescent tags often requires multiple reagents as well as multiple addition steps due to unique requirements of reagent storage and reaction milieus for different reagents.

Certain non-limiting embodiments of the present disclosure are directed to a diagnostic immunoassay reagent for use in particle enhanced agglutination immunoassays for an analyte (such as, but not limited to, a hapten). The diagnostic immunoassay reagent comprises a particle that has two antibodies or fragments thereof attached thereto. The first antibody or fragment thereof specifically binds to a portion (i.e., an epitope) of the analyte/hapten other than the portion (i.e., epitope) of the analyte/hapten to which the second antibody or fragment thereof specifically binds; that is, the first and second antibodies or fragments thereof bind to different (and substantially non-overlapping) epitopes of the analyte/hapten. In addition, binding of two of the diagnostic immunoassay reagents to the analyte/hapten results in particle enhanced agglutination for signal detection.

Any analyte (such as, but not limited to, a hapten/drug molecule) detectable by a sandwich immunoassay may be detectable by the reagents and methods disclosed herein. For example (but not by way of limitation), the hapten may be at least one of everolimus (for example, but not by way of limitation, RAD, Certican, ZORTRESS® (Novartis AG Corp., Basel, Switzerland)); sirolimus (for example, but not by way of limitation, rapamycin, RAPAMUNE® (Wyeth, LLC, Madison, N.J.)); tacrolimus (for example, but not by way of limitation, FK506, FR-900506, PROGRAF® (Astellas Pharma Inc., Tokyo, Japan)); and cyclosporine (for example, but not by way of limitation, cyclosporine A, cyclosporine B, cyclosporine C, cyclosporine D, cyclosporine E, cyclosporine F, cyclosporine G, cyclosporine H, cyclosporine I, and cyclosporine L). However, it will be understood that the present disclosure is not limited to use solely with haptens, and that larger analytes (such as, but not limited to, protein analytes) may also be detectable by the immunoassays disclosed herein.

Any type of particle known in the art for use in automated diagnostic immunoassays may be utilized in accordance with the present disclosure. In certain non-limiting embodiments, the particles should have an average diameter of at least about 0.02 microns and not more than about 100 microns. In some embodiments, the particles have an average diameter from about 0.05 microns to about 20 microns, or from about 0.3 microns to about 10 microns. The particle may be organic or inorganic, swellable or non-swellable, and porous or non-porous. In a particular (but not limiting) embodiment, the particle has a density approximating water, generally from about 0.7 g/mL to about 1.5 g/mL, and is composed of material that can be transparent, partially transparent, or opaque. The particles can be comprised of organic and inorganic polymers, latex particles, magnetic or non-magnetic particles, and the like. In some non-limiting examples, the particles are chrome particles or latex particles.

The polymer particles can be formed of addition or condensation polymers. The particles can also be derived from naturally occurring materials, naturally occurring materials that are synthetically modified, and synthetic materials. Among organic polymers of particular interest are polysaccharides, particularly cross-linked polysaccharides, such as (but not limited to) agarose, which is available as Sepharose; dextran, which is available as Sephadex and Sephacryl; cellulose; starch; and the like; addition polymers, such as polystyrene, polyvinyl alcohol, homopolymers and copolymers of derivatives of acrylate and methacrylate, particularly (but not limited to) esters and amides having free hydroxyl functionalities, and the like.

The particles will be readily dispersible in an aqueous medium and can be adsorptive or functionalizable so as to permit conjugation to two monoclonal antibodies for an immunosuppressant drug, either directly or indirectly through a linking group. When a linking group is utilized, in some non-limiting embodiments, the linking group may comprise about 2 to about 50 atoms, or 4 to about 30 atoms, not counting hydrogen and may comprise a chain of from 2 to about 30 atoms, or 3 to about 20 atoms, each independently selected from the group normally consisting of carbon, oxygen, sulfur, nitrogen, and phosphorous. Part or all of the linking group may be a portion of the molecule being linked to the immunosuppressant compound such as, but not limited to, an amino acid residue on a poly(amino acid), for example. In some examples, the linking group comprises an oxime functionality.

The number of heteroatoms in the linking group may be in the range from 0 to about 20, or 1 to about 15, or about 2 to about 10. The linking group may be aliphatic or aromatic. When heteroatoms are present, oxygen is normally present as oxo or oxy, bonded to carbon, sulfur, nitrogen or phosphorous, nitrogen is normally present as nitro, nitroso or amino, normally bonded to carbon, oxygen, sulfur or phosphorous; sulfur is analogous to oxygen; while phosphorous is bonded to carbon, sulfur, oxygen or nitrogen, usually as phosphonate and phosphate mono- or diester. Common functionalities in forming a covalent bond between the linking group and the molecule to be conjugated are alkylamine, amidine, thioamide, ether, urea, thiourea, guanidine, azo, thioether and carboxylate, sulfonate, and phosphate esters, amides and thioesters. One specific embodiment of a linking group comprising heteroatoms is an oxime functionality as mentioned above.

For the most part, when a linking group has a linking functionality (functionality for reaction with a moiety) such as, for example, a non-oxocarbonyl group including nitrogen and sulfur analogs, a phosphate group, an amino group, alkylating agent such as halo or tosylalkyl, oxy (hydroxyl or the sulfur analog, mercapto), oxocarbonyl (e.g., aldehyde or ketone), or active olefin such as a vinyl sulfone or α-, β-unsaturated ester, these functionalities are linked to amine groups, carboxyl groups, active olefins, alkylating agents, e.g., bromoacetyl. Where an amine and carboxylic acid or its nitrogen derivative or phosphoric acid are linked, amides, amidines, and phosphoramides are formed. Where mercaptan and activated olefin are linked, thioethers are formed. Where a mercaptan and an alkylating agent are linked, thioethers are formed. Where aldehyde and an amine are linked under reducing conditions, an alkylamine is formed. Where a ketone or aldehyde and a hydroxylamine (including derivatives thereof where a substituent is in place of the hydrogen of the hydroxyl group) are linked, an oxime functionality (═N—O—) is formed. Where a carboxylic acid or phosphate acid and an alcohol are linked, esters are formed. Various linking groups are well known in the art; see, for example, Cautrecasas (J. Biol. Chem. (1970) 245:3059).

In a particular (but non-limiting) embodiment of the diagnostic immunoassay reagent, the particle is a latex particle. In another particular (but non-limiting) embodiment of the diagnostic immunoassay reagent, each of the first and second antibodies or fragments thereof is attached to the particle via a linking group.

Any antibodies or fragments thereof known in the art or otherwise contemplatable by a person having ordinary skill in the art that are directed against the analyte/hapten/drug to be detected by the immunoassay method fall within the scope of the present disclosure. In a particular (but non-limiting) example, each of the antibodies or fragments thereof utilized in the diagnostic immunoassay reagent is a monoclonal antibody against tacrolimus, such as (but not limited to), the 14H04 or 1E2 monoclonal antibody clones disclosed previously in U.S. Pat. No. 8,586,322; or the 1H06 clone raised against the same immunogen as 14H04. The '322 patent provides the following definitions for these two antibody clones: (a) a monoclonal antibody that specifically binds to a portion (i.e., an epitope) of tacrolimus consisting essentially of the C29-C34 ring including the methoxy and hydroxyl substituents and C15 including the methoxy substituent (clone 14H04); (b) a monoclonal antibody that specifically binds to a portion (i.e., an epitope) of tacrolimus consisting essentially of the methoxy of the C10-C14 ring and C19-C27 of the C1-C26 ring including the C22 keto oxygen (clone 1E2); (c) a monoclonal antibody raised against an immunogen comprising an immunogenic carrier linked to tacrolimus at C22 (clones 14H04 and 1H06); (d) a monoclonal antibody raised against an immunogen comprising an immunogenic carrier linked to tacrolimus at C24 (clone 1E2); (e) a monoclonal antibody raised against an immunogen comprising an immunogenic carrier linked to tacrolimus at C32 (clone 1E2); and (f) a monoclonal antibody raised against an immunogen comprising an immunogenic carrier linked to tacrolimus at C24 and C32 (clone 1E2).

The 1H06 clone was raised against an immunogen containing FK coupled via oxime in 22 position and requires a C-22 coupled analog. This monoclonal antibody possesses a good cross-reactivity pattern and has an established affinity in the LOCI assay of KD <3*10⁻⁹.

In another particular (but non-limiting) embodiment, the antibody utilized in the assay is a monoclonal antibody against sirolimus. Non-limiting examples thereof include the IgG2aK clone, for which the immunogen is at the position C-32; and clones 3H9 (IgG1λ) and 165 (IgG2aK), which were raised against an immunogen comprising a C26 & C32 mixture. Thus, additional non-limiting examples of monoclonal antibodies that can be utilized in accordance with the present disclosure include (g) a monoclonal antibody raised against an immunogen comprising an immunogenic carrier linked to sirolimus at C32; and (h) a monoclonal antibody raised against a mixture of an immunogen comprising an immunogenic carrier linked to sirolimus at C26 and an immunogen comprising an immunogenic carrier linked to sirolimus at C32.

Certain non-limiting embodiments of the present disclosure are also directed to an aggregate of particles formed by interparticle bridging via an analyte/hapten molecule. These aggregates of particles include (A) an analyte/hapten; (B) a first antibody or fragment thereof attached to a particle, wherein the first antibody or fragment thereof is specifically bound to a portion (i.e., an epitope) of the analyte/hapten; and (C) a second antibody or fragment thereof, wherein the second antibody or fragment thereof is specifically bound to a portion (i.e., an epitope) of the analyte/hapten other than the portion/epitope of the analyte/hapten to which the first antibody or fragment thereof is bound. In addition, binding of binding of (B) and (C) to (A) results in particle enhanced agglutination.

The analyte/hapten and the particles of the aggregate of particles may be any of the analytes/haptens and particles, respectively, described or otherwise contemplated herein. In addition, each of the first and second antibodies or fragments thereof may be any of the antibodies or fragments thereof described or otherwise contemplated herein.

The second antibody or fragment thereof of (C) may be present in its “free” form (i.e., wherein the antibody/fragment is not directly attached to a particle), and as such, two diagnostic immunoassay reagents may be required to form the aggregate of particles. Alternatively, the second antibody or fragment thereof may be attached to a particle prior to combining with (A) and (B); in this embodiment, one or two diagnostic immunoassay reagents may be required to form the aggregate of particles, as described below. When the second antibody/fragment is attached to a particle, the particle of (C) may be the same type of particle as (B), or the particle of (C) may be different from the particle of (B). In addition, the particle(s) utilized in reagents (B) and/or (C) of the aggregate may be provided with both the first and the second antibodies or fragments thereof attached thereto. For example, the reagents (B) and (C) can be identical, and as such, only one type of diagnostic immunoassay reagent is required to form the aggregate of particles (where two copies of the same diagnostic immunoassay reagent are attached to different portions/epitopes of the analyte/hapten). Alternatively, the particles utilized in reagent(s) (B) and/or (C) may only contain the antibody/fragment thereof listed in (B) or (C), and as such, two different diagnostic immunoassay reagents are required to form the aggregate of particles.

Certain non-limiting embodiments of the present disclosure are also directed to a kit for the detection of the presence of an analyte (such as, but not limited to, a hapten) in a sample. The kit comprises one or more of any of the diagnostic immunoassay reagents as disclosed or otherwise contemplated herein. For example (but not by way of limitation), the kit comprises at least one diagnostic immunoassay reagent that includes: (1) a particle; (2) a first antibody or fragment thereof attached to the particle of (1); and (3) a second antibody or fragment thereof; wherein the first and second antibodies or fragments thereof specifically bind to different epitopes of an analyte/hapten present in the sample, thereby resulting in particle enhanced agglutination. When (3) is attached to (1), the kit will include only a single diagnostic immunoassay reagent in which (2) and (3) are both attached to (1). When (3) is not attached to (1), then the kit will include two diagnostic immunoassay reagents (one that contains (1) and (2), and another that contains (3)); in this embodiment, (3) may be present in the second diagnostic immunoassay reagent in its free form (i.e., not directly attached to a particle), or it may be attached to a different particle.

In certain non-limiting embodiments, the kits of the present disclosure further contain a pretreatment reagent. The pretreatment reagent may comprise at least one displacer/binding competitor or surfactant that functions to release the analyte/hapten from its endogenous binding proteins, as described in detail herein below.

Therapeutic drug monitoring (TDM) of immunophilin-binding drugs is particularly difficult, given that the binding of immunophilins endogenously present in biological samples will interfere with the assay. One method that has been utilized to attempt to overcome this interference is to add a substance that acts as a “displacer” by displacing the drug from its endogenous binding protein(s). Non-limiting examples of displacers can be found in U.S. Pat. No. 6,187,547, which discloses the use of these immunosuppressive drugs (ISD) with similar chemical structure to displace another ISD (i.e., the use of sirolimus to displace tacrolimus and vice versa); and U.S. Pat. No. 7,186,518, which discloses the use of a number of FK506 (tacrolimus) derivatives to displace FK506 from its endogenous binding proteins.

In addition, recently filed U.S. Application No. 62/754,913 (filed Nov. 2, 2018) discloses binding competitors that are used to displace the hapten/drug from its endogenous binding proteins/immunophilin complexes and that do not significantly bind to the first and second antibodies or fragments thereof that are utilized in the immunoassay. As such, the pretreatment reagent of the present disclosure may specifically include any of the binding competitors disclosed in the '913 application.

For example (but not by way of limitation), when the hapten to be detected is tacrolimus, the pretreatment reagent may comprise sirolimus or everolimus. Alternatively, when the hapten to be detected is sirolimus or everolimus, the pretreatment reagent may comprise at least one binding competitor selected from the compounds represented by Formulas IV, V, VI, and VII below.

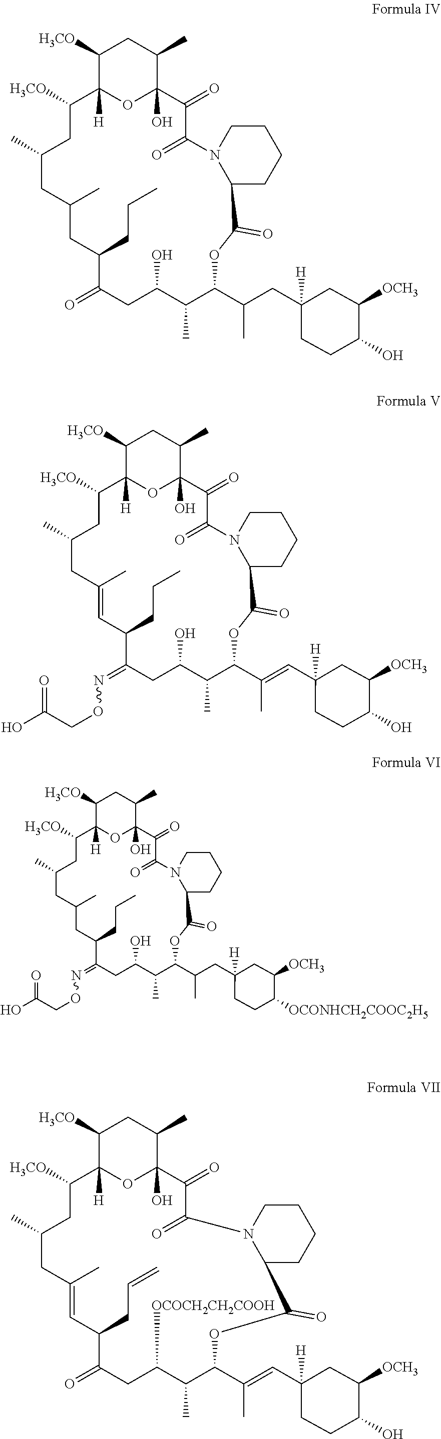

The assay components/reagents of the compositions/kits/methods may be provided in any form that allows them to function in accordance with the present disclosure. For example, but not by way of limitation, each of the reagents may be provided in liquid form and disposed in bulk and/or single aliquot form within the kit. Alternatively, in a particular (but non-limiting) embodiment, one or more of the reagents may be disposed in the kit in the form of a single aliquot lyophilized reagent. The use of dried reagents in microfluidics devices is described in detail in U.S. Pat. No. 9,244,085 (Samproni), the entire contents of which are hereby expressly incorporated herein by reference.

In addition to the assay components/reagents described in detail herein above, the kits may further contain other reagent(s) for conducting any of the particular assays described or otherwise contemplated herein. The nature of these additional reagent(s) will depend upon the particular assay format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the cross-reactivity and stability of the components/reagents. In addition, the kit may include a microfluidics device in which the components/reagents are disposed.

The relative amounts of the various components/reagents in the kits can vary widely to provide for concentrations of the components/reagents that substantially optimize the reactions that need to occur during the assay methods and further to optimize substantially the sensitivity of an assay. Under appropriate circumstances, one or more of the components/reagents in the kit can be provided as a dry powder, such as a lyophilized powder, and the kit may further include excipient(s) for dissolution of the dried reagents; in this manner, a reagent solution having the appropriate concentrations for performing a method or assay in accordance with the present disclosure can be obtained from these components. Positive and/or negative controls may also be included with the kit. In addition, the kit can further include a set of written instructions explaining how to use the kit. A kit of this nature can be used in any of the methods described or otherwise contemplated herein.

Certain non-limiting embodiments of the present disclosure are further directed to a method of detecting the presence of an analyte (such as, but not limited to, a hapten) in a sample. The method comprises the steps of: (i) exposing the sample to any of the pretreatment reagents disclosed or otherwise contemplated herein to release the analyte/hapten from endogenous binding proteins and provide a pretreated sample; (ii) mixing the pretreated sample formed in (i) with any of the one or more diagnostic immunoassay reagents disclosed or otherwise contemplated herein to form a mixture; (iii) incubating the mixture formed in (ii) under conditions for binding of the diagnostic immunoassay reagent to the analyte/hapten present in the sample, thereby resulting in particle enhanced agglutination; and (iv) detecting a level of particle enhanced agglutination present in the incubated mixture and correlating the level of particle enhanced agglutination to a level of analyte/hapten present in the sample.

In a particular (but non-limiting) embodiment, only a single type of diagnostic immunoassay reagent is supplied in step (ii), and that reagent contains both first and second antibodies/fragments attached to a single particle, as described herein above. In this method, two copies of the same diagnostic immunoassay reagent are attached to different portions/epitopes of the analyte/hapten to form the aggregate of particles that causes agglutination.

Alternatively, two types of diagnostic immunoassay reagents are supplied in step (ii): the first reagent includes the first antibody/fragment attached to a particle, and the second reagent includes the second antibody/fragment, either in its free form (i.e., not directly attached to a particle) or attached to a particle. In this embodiment, two different diagnostic immunoassay reagents must bind to a single analyte/hapten to form the aggregate of particles. This embodiment of the method includes the following steps: (i) exposing the sample to a pretreatment reagent (as described or otherwise contemplated herein) to release the analyte/hapten from endogenous binding proteins and provide a pretreated sample; (ii) mixing the pretreated sample formed in (i) with two diagnostic immunoassay reagents to form a mixture, wherein the first diagnostic immunoassay reagent comprises a first particle having a first antibody or fragment thereof attached thereto, and the second diagnostic immunoassay reagent comprises a second antibody or fragment thereof (either in its free form or attached to a particle), wherein the first antibody or fragment thereof specifically binds to a portion/epitope of the analyte/hapten other than the portion/epitope of the analyte/hapten to which the second antibody or fragment thereof specifically binds (and wherein the first and second antibodies/fragments and particles are any of the antibodies/fragments or particles, respectively, described or otherwise contemplated herein); (iii) incubating the mixture formed in (ii) under conditions for binding of the two diagnostic immunoassay reagents to the analyte/hapten present in the sample, thereby resulting in particle enhanced agglutination; and (iv) detecting a level of particle enhanced agglutination present in the incubated mixture and correlating the level of particle enhanced agglutination to a level of analyte/hapten present in the sample.

Any methods of detecting agglutination known in the art or otherwise contemplatable to a person having ordinary skill in the art fall within the scope of the present disclosure and can be utilized with any of the methods disclosed or otherwise contemplated herein. In certain non-limiting embodiments, agglutination can be detected turbidimetrically or metallometrically by methods well known in the art.

In particular (but non-limiting) embodiments, particle enhanced agglutination immunoassays involve the measurement of intensity of scattered light and the measurement of absorbance. Non-limiting examples of analytical steps performed in particle enhanced agglutination immunoassays are described, for example, in US Patent Application Publication No. 2019/0154674. In particular, the '674 publication teaches that these steps can include: mixing a analyte-containing sample with insoluble carrier particles carrying a binding partner(s) for the analyte to prepare a mixed solution; determining a variation (i) in intensity of light scattered from the mixed solution based on a difference in intensity of scattered light between first and second time points; determining a variation (ii) in absorbance of the mixed solution based on a difference in absorbance between third and fourth time points; and correlating the determined variation (i) in intensity of scattered light and the determined variation (ii) in absorbance with an amount of the analyte present in the sample using a calibration curve plotted based on the variation in intensity of scattered light and a calibration curve plotted based on the variation in absorbance.

However, the present disclosure is not limited to any of the examples provided herein above; any other set of analytical steps utilizable in performing particle enhanced agglutination immunoassays in an automated environment that are known in the art or otherwise contemplatable by a person having ordinary skill in the art also fall within the scope of the present disclosure.

### EXAMPLE

An Example is provided hereinbelow. However, the present disclosure is to be understood to not be limited in its application to the specific experimentation, results, and laboratory procedures disclosed herein. Rather, the Example is simply provided as one of various embodiments and is meant to be exemplary, not exhaustive.

### Preparation of Diagnostic Immunoassay Reagent

There are three possible ways of coating the latex particle with antibodies using two anti-hapten antibodies (with two anti-FK506 antibodies being used in this example for purposes of illustration only). First, two latex particle preparations are prepared: one is latex particles coated with 1E2, and the other is latex particles coated with 14H04. Second, a latex particle is coated with either 1E2 or 14H04, and the second antibody remains in its free form. Third, a latex particle is coated with a mixture of 1E2 and 14H04. Both preparation methods could be used in a similar fashion in the assay, but only the latex particle coated with the mixture of 14H04 and 1E2 is used in the assay below.

### Immunoassay Format Using Particle Enhanced Agglutination

The assay sequence includes the following steps: (a) a whole blood sample is mixed with a pretreatment reagent; (b) the latex particles produced as above are added to the mixture and incubated; and (c) the drug signal is detected.

The pretreatment reagent used in step (a) contains a displacer/surfactant for the purpose of freeing up FK506 drug from the endogenous binding proteins. After the latex particles are added in step (b), the reaction mixture is allowed to incubate during this process, and then reads can be taken in step (c) to detect particle agglutination.

Thus, in accordance with the present disclosure, there have been provided compositions, kits, and devices, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

1. A diagnostic immunoassay reagent, comprising:
a particle;
a first antibody or fragment thereof attached to the particle, wherein the first antibody specifically binds to a portion of a hapten; and
a second antibody or fragment thereof attached to the particle, wherein the second antibody binds to a portion of the hapten other than the portion of the hapten to which the first antibody specifically binds;
whereby binding of hapten to the diagnostic immunoassay reagent results in particle enhanced agglutination for signal detection.

3. The diagnostic immunoassay reagent of claim 2, wherein the hapten is selected from the group consisting of tacrolimus, everolimus, sirolimus, and cyclosporine.

4. The diagnostic immunoassay reagent of claim 1, wherein the particle is a latex particle.

5. The diagnostic immunoassay reagent of claim 1, wherein each of the first and second antibodies or fragments thereof is selected from the group consisting of:
(a) a monoclonal antibody or fragment thereof that specifically binds to a portion of tacrolimus consisting essentially of the C29-C34 ring including the methoxy and hydroxyl substituents and C15 including the methoxy substituent;
(b) a monoclonal antibody or fragment thereof that specifically binds to a portion of tacrolimus consisting essentially of the methoxy of the C10-C14 ring and C19-C27 of the C1-C26 ring including the C22 keto oxygen;
(c) a monoclonal antibody or fragment thereof raised against an immunogen comprising an immunogenic carrier linked to tacrolimus at C22;
(d) a monoclonal antibody or fragment thereof raised against an immunogen comprising an immunogenic carrier linked to tacrolimus at C24;
(e) a monoclonal antibody or fragment thereof raised against an immunogen comprising an immunogenic carrier linked to tacrolimus at C32;
(f) a monoclonal antibody or fragment thereof raised against an immunogen comprising an immunogenic carrier linked to tacrolimus at C24 and C32;
(g) a monoclonal antibody or fragment thereof raised against an immunogen comprising an immunogenic carrier linked to sirolimus at C32; and
(h) a monoclonal antibody or fragment thereof raised against a mixture of an immunogen comprising an immunogenic carrier linked to sirolimus at C26 and an immunogen comprising an immunogenic carrier linked to sirolimus at C32.

6. An aggregate of particles, comprising:
(A) a hapten;
(B) a first antibody or fragment thereof attached to a particle, wherein the first antibody or fragment thereof is specifically bound to a portion of the hapten; and
(C) a second antibody or fragment thereof, wherein the second antibody or fragment thereof is specifically bound to a portion of the hapten other than the portion of the hapten to which the first antibody or fragment thereof is bound; and
wherein binding of (B) and (C) to (A) results in particle enhanced agglutination.

7. The aggregate of particles of claim 6, wherein the second antibody or fragment thereof is not directly attached to a particle.

8. The aggregate of particles of claim 6, wherein the second antibody or fragment thereof is attached to a particle.

9. The aggregate of particles of claim 8, wherein the second antibody or fragment thereof is attached to the particle of (B).

10. The aggregate of particles of claim 6, wherein the hapten is selected from the group consisting of tacrolimus, everolimus, sirolimus, and cyclosporine.

11. The aggregate of particles of claim 6, wherein the particle is a latex particle.

12. The aggregate of particles of claim 6, wherein each of the first and second antibodies or fragments thereof is selected from the group consisting of:
(a) a monoclonal antibody or fragment thereof that specifically binds to a portion of tacrolimus consisting essentially of the C29-C34 ring including the methoxy and hydroxyl substituents and C15 including the methoxy substituent;
(b) a monoclonal antibody or fragment thereof that specifically binds to a portion of tacrolimus consisting essentially of the methoxy of the C10-C14 ring and C19-C27 of the C1-C26 ring including the C22 keto oxygen;
(c) a monoclonal antibody or fragment thereof raised against an immunogen comprising an immunogenic carrier linked to tacrolimus at C22;
(d) a monoclonal antibody or fragment thereof raised against an immunogen comprising an immunogenic carrier linked to tacrolimus at C24;
(e) a monoclonal antibody or fragment thereof raised against an immunogen comprising an immunogenic carrier linked to tacrolimus at C32;
(f) a monoclonal antibody or fragment thereof raised against an immunogen comprising an immunogenic carrier linked to tacrolimus at C24 and C32;
(g) a monoclonal antibody or fragment thereof raised against an immunogen comprising an immunogenic carrier linked to sirolimus at C32; and
(h) a monoclonal antibody or fragment thereof raised against a mixture of an immunogen comprising an immunogenic carrier linked to sirolimus at C26 and an immunogen comprising an immunogenic carrier linked to sirolimus at C32.

13. A kit for a diagnostic immunoassay, the kit comprising:
at least one diagnostic immunoassay reagent comprising:
(1) a particle;
(2) a first antibody or fragment thereof attached to the particle of (1); and
(3) a second antibody or fragment thereof; and
wherein the first and second antibodies or fragments thereof specifically bind to different epitopes of a hapten present in the sample, thereby resulting in particle enhanced agglutination; and
a pretreatment reagent comprising a displacer for releasing the hapten from endogenous binding proteins.

14. The kit of claim 13, wherein (3) is not directly attached to a particle.

15. The kit of claim 13, wherein (3) is attached to (1).

16. The kit of claim 13, wherein the hapten is selected from the group consisting of tacrolimus, everolimus, sirolimus, and cyclosporine.

17. The kit of claim 13, wherein the particle of the diagnostic immunoassay reagent is a latex particle.

18. The kit of claim 13, wherein each of the first and second antibodies or fragments thereof is selected from the group consisting of:
(a) a monoclonal antibody or fragment thereof that specifically binds to a portion of tacrolimus consisting essentially of the C29-C34 ring including the methoxy and hydroxyl substituents and C15 including the methoxy substituent;
(b) a monoclonal antibody or fragment thereof that specifically binds to a portion of tacrolimus consisting essentially of the methoxy of the C10-C14 ring and C19-C27 of the C1-C26 ring including the C22 keto oxygen;
(c) a monoclonal antibody or fragment thereof raised against an immunogen comprising an immunogenic carrier linked to tacrolimus at C22;
(d) a monoclonal antibody or fragment thereof raised against an immunogen comprising an immunogenic carrier linked to tacrolimus at C24;
(e) a monoclonal antibody or fragment thereof raised against an immunogen comprising an immunogenic carrier linked to tacrolimus at C32;
(f) a monoclonal antibody or fragment thereof raised against an immunogen comprising an immunogenic carrier linked to tacrolimus at C24 and C32;
(g) a monoclonal antibody or fragment thereof raised against an immunogen comprising an immunogenic carrier linked to sirolimus at C32; and
(h) a monoclonal antibody or fragment thereof raised against a mixture of an immunogen comprising an immunogenic carrier linked to sirolimus at C26 and an immunogen comprising an immunogenic carrier linked to sirolimus at C32.

19. The kit of claim 13, wherein the pretreatment reagent comprises at least one binding competitor that displaces the hapten from its endogenous binding proteins, wherein the binding competitor is at least one of:
(a) sirolimus;
(b) everolimus;
(c) the compound represented by Formula IV: 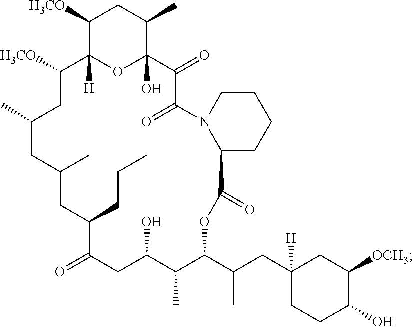 [SMILES: CCC[C@@H]1CC(C)C[C@H](C)C[C@H](OC)[C@H]2O[C@@](O)(C(=O)C(=O)N3CCCC[C@H]3C(=O)O[C@H](C(C)C[C@@H]3CC[C@@H](O)[C@H](OC)C3)[C@H](C)[C@@H](O)CC1=O)[C@H](C)C[C@@H]2OC]
(d) the compound represented by Formula V: 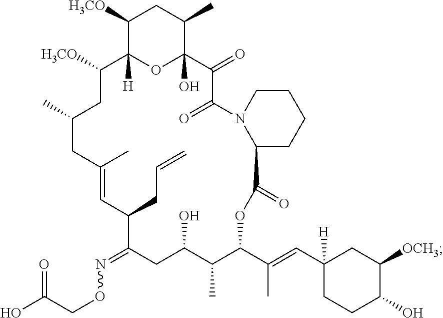 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=NOCC(=O)O)[C@H](C)C[C@@H]2OC]
(e) the compound represented by Formula VI: 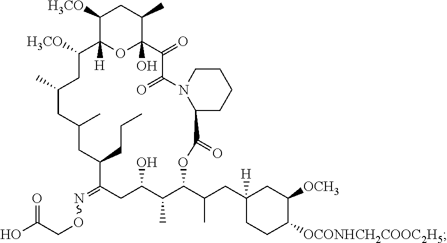
or
(f) the compound represented by Formula VII:

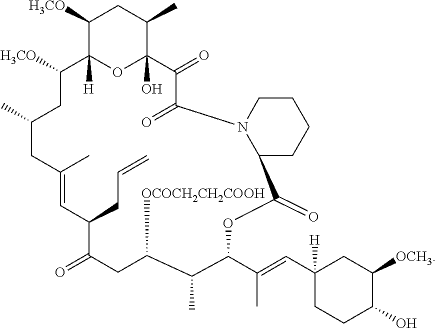

20. A method of detecting the presence of a hapten in a sample, the method comprising the steps of:
(i) exposing the sample to a pretreatment reagent to release the hapten from endogenous binding proteins and provide a pretreated sample;
(ii) mixing the pretreated sample formed in (i) with at least one diagnostic immunoassay reagent to form a mixture, wherein the at least one diagnostic immunoassay reagent comprises a particle, a first antibody or fragment thereof attached to the particle, and a second antibody or fragment thereof, wherein the first antibody or fragment thereof specifically binds to a portion of a hapten other than the portion of the hapten to which the second antibody or fragment thereof specifically binds;
(iii) incubating the mixture formed in (ii) under conditions for binding of the at least one diagnostic immunoassay reagent to the hapten present in the sample, thereby resulting in particle enhanced agglutination; and
(iv) detecting a level of particle enhanced agglutination present in the incubated mixture and correlating the level of particle enhanced agglutination to a level of hapten present in the sample.

21. The method of claim 20, wherein the second antibody or fragment thereof present in the at least one diagnostic immunoassay reagent is not directly attached to a particle.

22. The method of claim 20, wherein the second antibody or fragment thereof present in the at least one diagnostic immunoassay reagent is attached to a particle.

23. The method of claim 20, wherein the first and second antibodies or fragments thereof present in the at least one diagnostic immunoassay reagent are attached to the same particle.

24. The method of claim 20, wherein the hapten is selected from the group consisting of tacrolimus, everolimus, sirolimus, and cyclosporine.

25. The method of claim 20, wherein the particle of the at least one diagnostic immunoassay reagent is a latex particle.

26. The method of claim 20, wherein each of the first and second antibodies or fragments thereof of the at least one diagnostic immunoassay reagent is selected from the group consisting of:
(a) a monoclonal antibody or fragment thereof that specifically binds to a portion of tacrolimus consisting essentially of the C29-C34 ring including the methoxy and hydroxyl substituents and C15 including the methoxy substituent;
(b) a monoclonal antibody or fragment thereof that specifically binds to a portion of tacrolimus consisting essentially of the methoxy of the C10-C14 ring and C19-C27 of the C1-C26 ring including the C22 keto oxygen;
(c) a monoclonal antibody or fragment thereof raised against an immunogen comprising an immunogenic carrier linked to tacrolimus at C22;
(d) a monoclonal antibody or fragment thereof raised against an immunogen comprising an immunogenic carrier linked to tacrolimus at C24;
(e) a monoclonal antibody or fragment thereof raised against an immunogen comprising an immunogenic carrier linked to tacrolimus at C32;
(f) a monoclonal antibody or fragment thereof raised against an immunogen comprising an immunogenic carrier linked to tacrolimus at C24 and C32;
(g) a monoclonal antibody or fragment thereof raised against an immunogen comprising an immunogenic carrier linked to sirolimus at C32; and
(h) a monoclonal antibody or fragment thereof raised against a mixture of an immunogen comprising an immunogenic carrier linked to sirolimus at C26 and an immunogen comprising an immunogenic carrier linked to sirolimus at C32.

27. The method of claim 20, wherein the pretreatment reagent comprises a binding competitor that displaces the hapten from its endogenous binding proteins, wherein the binding competitor is at least one of:
(a) sirolimus;
(b) everolimus;
(c) the compound represented by Formula IV: 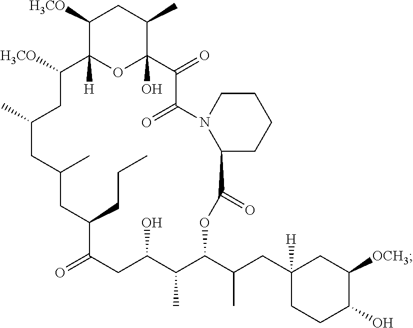 [SMILES: CCC[C@@H]1CC(C)C[C@H](C)C[C@H](OC)[C@H]2O[C@@](O)(C(=O)C(=O)N3CCCC[C@H]3C(=O)O[C@H](C(C)C[C@@H]3CC[C@@H](O)[C@H](OC)C3)[C@H](C)[C@@H](O)CC1=O)[C@H](C)C[C@@H]2OC]
(d) the compound represented by Formula V: 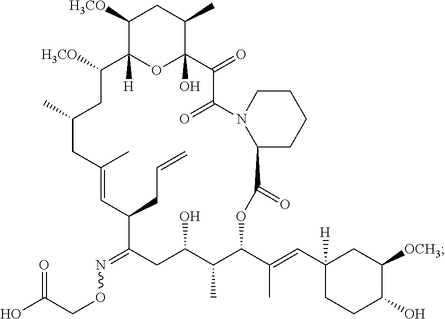 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=NOCC(=O)O)[C@H](C)C[C@@H]2OC]
(e) the compound represented by Formula VI: 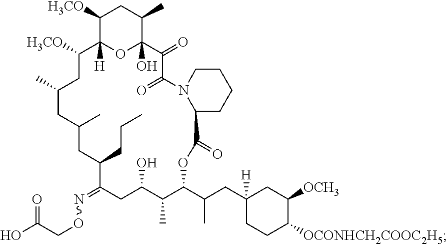
or
(f) the compound represented by Formula VII:

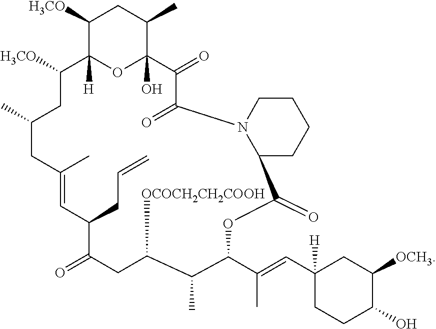

28. A method of detecting the presence of a hapten in a sample, the method comprising the steps of:
(i) exposing the sample to a pretreatment reagent to release the hapten from endogenous binding proteins and provide a pretreated sample;
(ii) mixing the pretreated sample formed in (i) with at least one diagnostic immunoassay reagent to form a mixture, wherein the at least one diagnostic immunoassay reagent comprises a particle, a first antibody or fragment thereof attached to the particle, and a second antibody or fragment thereof, wherein the first antibody or fragment thereof specifically binds to a portion of the hapten other than the portion of the hapten to which the second antibody or fragment thereof specifically binds, and wherein:
(a) the second antibody or fragment thereof present in the at least one diagnostic immunoassay reagent is not directly attached to a particle; or
(b) the first and second antibodies or fragments thereof present in the at least one diagnostic immunoassay reagent are attached to the same particle;
(iii) incubating the mixture formed in (ii) under conditions for binding of the at least one diagnostic immunoassay reagent to the hapten present in the sample, thereby resulting in particle enhanced agglutination; and
(iv) detecting a level of particle enhanced agglutination present in the incubated mixture and correlating the level of particle enhanced agglutination to a level of hapten present in the sample.
